(19) United States
(12) Patent Application Publication
(10) Pub. No.: US 2016/0100931 A1
(43) Pub. Date: Apr. 14, 2016

(54) METHODS AND SYSTEMS FOR FORMING UNICOMPARTMENTAL AND UNICONDYLAR KNEE RESURFACING IN CONJUNCTION WITH CRUCIATE LIGAMENT REPLACEMENT CONCOMITANTLY

(71) Applicant: Arthrex, Inc., Naples, FL (US)

(72) Inventors: Robert K. Sluss, Naples, FL (US); Jacob A. Jolly, Naples, FL (US); Vipool K. Goradia, Midlothian, VA (US); Thomas M. Deberardino, Avon, CT (US)

(21) Appl. No.: 14/513,904

(22) Filed: Oct. 14, 2014

(51) Int. Cl. A61F 2/08; A61B 17/16

(52) U.S. Cl. CPC A61F 2/0811; A61B 17/1675

(57) ABSTRACT

Instruments, implants and methods to concomitantly perform unicompartmental knee resurfacing and cruciate ligament reconstruction procedures. Unicondylar knee resurfacing is conducted with concomitant knee ligament reconstruction (such as, for example, GraftLink® All-Inside ACL Reconstruction using TightRope® ABS) and employing retrograde drilling. Retrograde drilling, which starts at the level of the resurfacing implants and travels away from the joint line and resurfacing implants, allows for more precise placement of the drill holes and also the drill trajectory, to avoid undermining the resurfacing implants. Retrograde drills also allow the surgeon to see the drill hole footprint at the joint line and resurfacing implant level more accurately, so the surgeon can place the starting point of the drill hole away from the resurfacing implants.

### FIELD OF THE INVENTION

The present invention relates to surgical devices and, in particular, to devices and methods for arthroscopic surgeries.

### BACKGROUND OF THE INVENTION

Partial knee replacement surgery, also called unicompartmental knee arthroplasty, is routinely considered for the treatment of osteoarthritis of the knee joint. Partial knee replacement surgery has generated significant interest because it entails a smaller incision and faster recovery than traditional total joint replacement surgery.

Knee instability caused by either an anterior or posterior cruciate ligament laxity or compromise of the actual ligament due to injury is a contraindication to performing unicompartmental or unicondylar knee resurfacing procedures because of the resultant increased stresses placed upon the resurfacing implants. Having to stage these procedures increases the patient risk to morbidities associate with multiple surgical procedures.

Traditional methods for reconstructing the cruciate ligaments include antegrade drilling of tunnels through the tibial plateau and the femur to accept a tissue graft and implants to fixate the graft securely while it heals.

Unicompartmental and unicondylar knee resurfacing implants are traditionally cemented to a portion of the femoral condyle and tibial plateau bone after removal of the diseased portion of bone and cartilage to provide pain relief and restoration of limb alignment and compartmental kinematics. The implants are located in one of the compartments of the knee that are usually in close proximity to the tunnels drilled for graft placement during cruciate ligament reconstruction procedures.

Undermining the femoral or tibial implants for unicompartmental resurfacing could lead to interruption of the cement mantle and bone and subsequent subsidence of the implants or fracture of the bone leading to failure. Even with the use of targeting guides it has been demonstrated that accurately guiding the trajectory of the drills used to create antegrade drill holes to accept the graft is difficult to control and replicate and could lead to undermining the tibial or femoral bone on which the resurfacing implants will be attached.

There is a need for providing surgeons with instruments, implants, kits and systems to concomitantly perform unicompartmental knee resurfacing and knee ligament reconstruction procedures (such as ACL reconstruction, for example) without compromising either implant construct, as well as reducing overall morbidities associated with multiple staged surgical procedures. Also needed are methods and techniques for simultaneously-conducted cruciate ligament reconstruction and unicompartmental knee resurfacing procedures that provide a functional ACL with normal kinematics of the knee after unicondylar resurfacing/replacement. Methods and techniques that minimize graft damage during bone preparation and enable correct graft placement and tensioning with the joint space restored are also needed.

### SUMMARY OF THE INVENTION

The present invention fulfills the above needs and objectives by providing improved systems and surgical techniques for arthroscopic procedures. The invention provides medical personnel with instruments and implants to concomitantly perform unicompartmental knee resurfacing and cruciate ligament reconstruction procedures without compromising either implant construct as well as reducing overall morbidities associated with multiple staged surgical procedures. The methods and systems of the present invention allow forming unicompartmental and unicondylar knee resurfacing in conjunction with cruciate ligament replacement concomitantly.

Unicondylar knee resurfacing is conducted with concomitant knee ligament reconstruction (such as, for example, GraftLink® All-Inside ACL Reconstruction using TightRope® ABS) and employing retrograde drilling. Retrograde drilling, which starts at the level of the resurfacing implants and travels away from the joint line and resurfacing implants, allows for more precise placement of the drill holes and also the drill trajectory, to avoid undermining the resurfacing implants. Retrograde drills also allow the surgeon to see the drill hole footprint at the joint line and resurfacing implant level more accurately, so the surgeon can place the starting point of the drill hole away from the resurfacing implants.

Other features and advantages of the present invention will become apparent from the following description of the invention.

### BRIEF DESCRIPTION OF THE DRAWINGS

FIG. 1 illustrates an exemplary schematic view of a knee undergoing an ACL replacement and unicompartmental knee arthoplasty (UKA) concomitantly.

FIGS. 2-25 illustrate subsequent steps of a method of conducting unicompartmental and unicondylar knee resurfacing in conjunction with cruciate ligament replacement concomitantly, and according to an exemplary embodiment of the present invention.

FIG. 26 illustrates a final knee repair by the method of FIGS. 2-25 and according to an exemplary embodiment of the present invention.

FIGS. 27-35 illustrate a method of forming an exemplary tissue construct employed in the method of FIGS. 2-25.

FIGS. 36-38 illustrate various views of UKA implant components employed with the method of FIGS. 2-25

### DETAILED DESCRIPTION OF THE EMBODIMENTS

The present invention provides advanced preservation and restoration techniques that extend a surgeon's ability to provide a broader continuum of care to treat a patient's knee pathology. The systems and methods of the invention allow the surgeons to offer patients an advanced, yet simple, option to treat the progression of joint degeneration and the underlying injuries they may encounter throughout their lifetime.

An exemplary method of conducting ligament reconstruction concomitantly with unicondylar resurfacing comprises inter alia the steps of: (i) forming femoral and tibial tunnels or sockets, at least the tibial tunnel or socket being formed by retrograde drilling; (ii) installing femoral and tibial components of a knee implant; and (iii) securing a tissue construct within the femoral and tibial tunnels or sockets.

In an exemplary embodiment, the tissue construct is an adjustable suture-button construct that is provided with at least one adjustable, knotless, flexible loop member having an adjustable length, two splices that are interconnected, and at least one button that is adapted for engagement with the adjustable, knotless, flexible loop member and that engages with a ligament.

In another embodiment, the tissue construct is a knotless, adjustable suture loop/button construct with first and second buttons that may have similar or different configuration, a continuous loop of flexible material attached to each button, each of the loops having an adjustable length to allow positioning of a tissue (e.g., ligament) within the tibia and femoral tunnels/sockets, and wherein each loop is provided with two discrete splices that allow adjustment in one direction while locking the construct in the opposite direction, and tissue (soft tissue, ligament, graft, BTB, or combinations thereof) securely attached to each of the two loops for positioning. In an exemplary only embodiment, the tissue construct is a presutured GraftLink® construct employed for an all-inside ACL reconstruction with TightRope® ABS, as detailed and described in U.S. Pat. No. 8,591,578 issued Nov. 26, 2013 (entitled ADJUSTABLE SUTURE-BUTTON CONSTRUCTS FOR LIGAMENT RECONSTRUCTION), the disclosure of which is incorporated by reference in its entirety herewith.

In an exemplary embodiment, the tissue construct is a GraftLink® used in combination with a FlipCutter® that allows independent femoral and tibial retrodrilling to create sockets, while maintaining the cortices to maximize fixation. A single hamstring harvest reduces morbidity and preserves strength. Two suspension TightRope® fixation implants, with a proprietary four-point locking system, make GraftLink® an innovative, reproducible ACL reconstruction.

Another exemplary method of conducting knee ligament reconstruction (for example, ACL reconstruction) concomitantly with unicondylar resurfacing comprises inter alia the steps of: (i) forming femoral and tibial tunnels or sockets, at least the tibial tunnel or socket being formed by retrograde drilling employing a retrocutter, a flip cutter or a dual-sided rotary drill cutter that is configured to cut in both directions, antegrade and retrograde; (ii) resecting tibia at least in horizontal and vertical directions; (iii) resecting femur at least distally and posteriorly; (iv) assessing the fit and position of tibial and femoral implants by employing at least one of a D-ring tibial trial, tibial bearing trials and femoral component trials; and (v) simultaneously (a) implanting the tibial and femoral implants within the tibia and femur and (b) implanting a GraftLink® construct within the femoral and tibial tunnels or sockets. In an exemplary-only embodiment, step (v) further comprises the steps of: installing the tibial trial bearing into the tibial tray; then, shuttling the Graftlink® construct into the femoral and tibial tunnels or sockets in an all-inside intraarticular manner; waiting for the UKA cement to dry; and then replacing the trial bearing with the final bearing of the tibial implant.

Another exemplary method of conducting ACL ligament reconstruction concomitantly with UKA comprises inter alia the steps of (i) forming femoral and tibial tunnels or sockets, at least the tibial tunnel or socket being formed by retrograde drilling employing a retrocutter, a flip cutter or a dual-sided rotary drill cutter that is configured to cut in both directions, antegrade and retrograde; (ii) resecting tibia in horizontal and vertical directions, and next and lateral to, the articular opening of the tibial tunnel or socket; (iii) measuring the flexion space by employing a spacer block into the leg compartment; (iv) resecting the femur at least distally and posteriorly with a distal cutting block and a posterior cutting block; (v) creating anterior and posterior lug holes using a femoral step drill; (vi) assessing the fit and position of tibial and femoral implants by employing at least one of a D-ring tibial trial, tibial bearing trials and femoral component trials; and (vii) simultaneously implanting the tibial and femoral implants within the tibia and femur and implanting a GraftLink® construct within the femoral and tibial tunnels or sockets by conducting the steps of (a) installing the tibial trial bearing into the tibial tray; (b) then, implanting the GraftLink® construct into the tibial and femoral sockets; (c) then, waiting for the UKA cement to harden; and (d) then, replacing trial bearing with final poly bearing of the tibial implant.

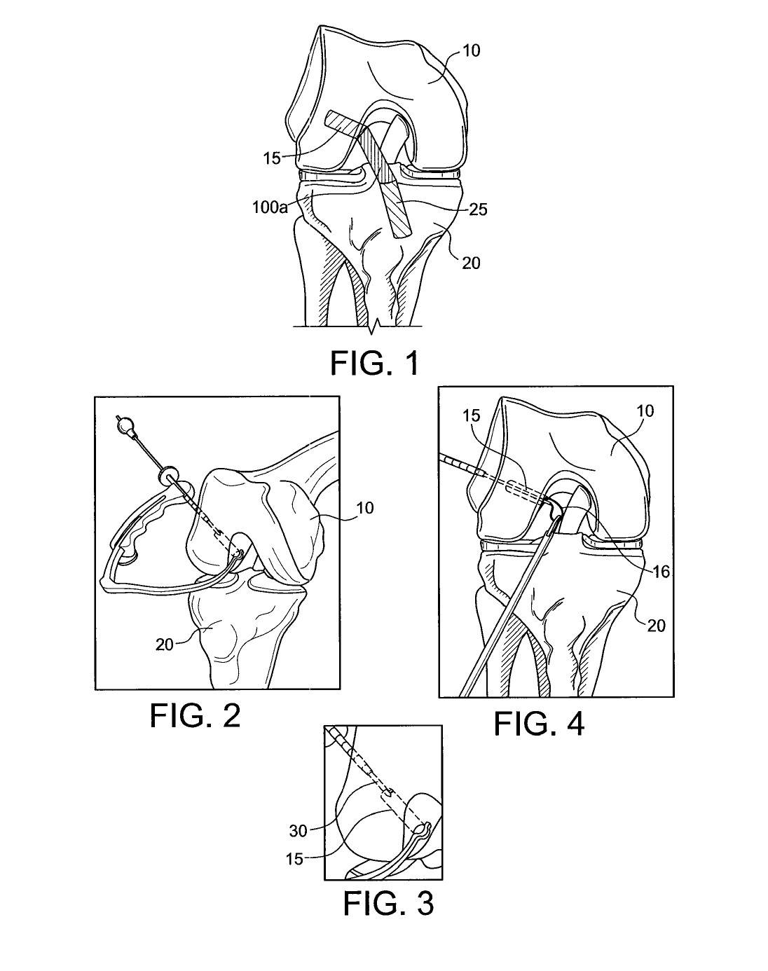

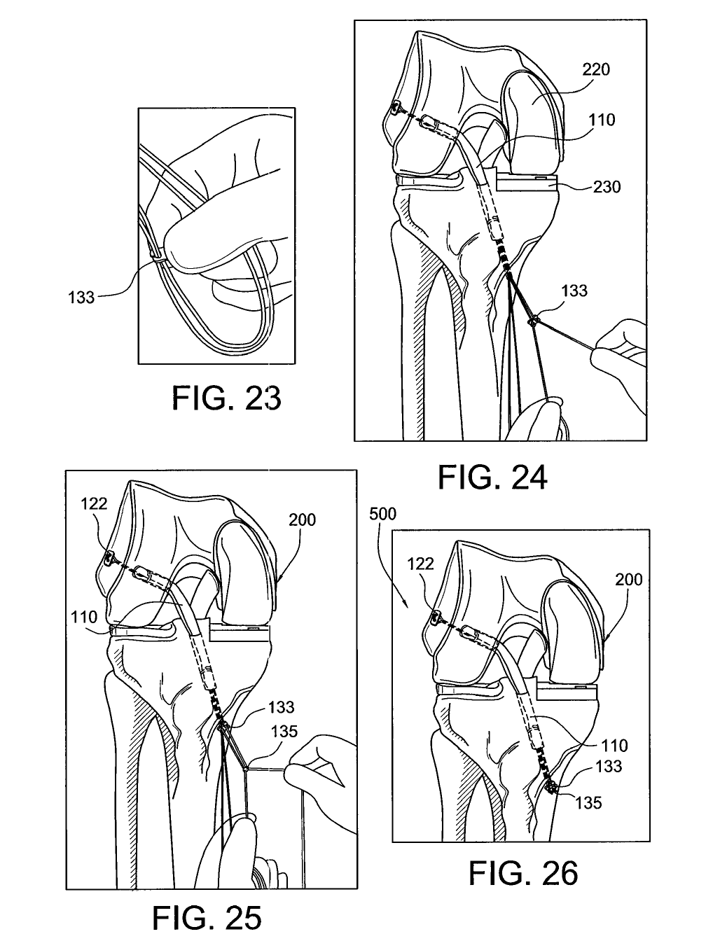

Referring now to the drawings, where like elements are designated by like reference numerals, FIGS. 1-38 illustrate exemplary systems and steps of a method of conducting ligament reconstruction concomitant with UKA or unicondylar resurfacing.

FIG. 1 illustrates a schematic view of ligament 100a (for example, ACL 100a) to be replaced in a patient's knee by a method of the present invention and as detailed below. Ligament 100a extends between tibia 20 and femur 10. The length from the end of femoral socket 15 to the end of tibial socket 25 should be at least 10 mm longer than the graft 100a to ensure that the graft can be tensioned fully. An example is a 70 mm graft length. Assuming a maximum intraarticular length of 30 mm, there will be approximately 20 mm of graft in the femoral and tibial sockets 15, 25. Drill the femur 20 mm deep and the tibia approximately 30 mm deep to allow an extra 10 mm for tensioning.

FIGS. 2-4 illustrate arthroscopic femoral socket preparation for medial UKA. For medial portal drilling, use the TightRope® Drill Pin, Transportal ACL Guides and Low Profile Reamers. Note the intraosseous length from the TightRope® Drill Pin. After socket drilling, pass a suture with the TightRope® Drill Pin for later graft passing. An exemplary method includes forming the femoral socket by employing a retrograde cutter 30, for example FlippCutter® 30. After “flipcutting,” pass a FiberStick™ suture 16 through the stepped drill sleeve and dock for later graft passing. When performing a lateral unicondylar knee replacement procedure, drill the femoral tunnel 15 after preparing the lateral femoral condyle for the UKA femoral implant.

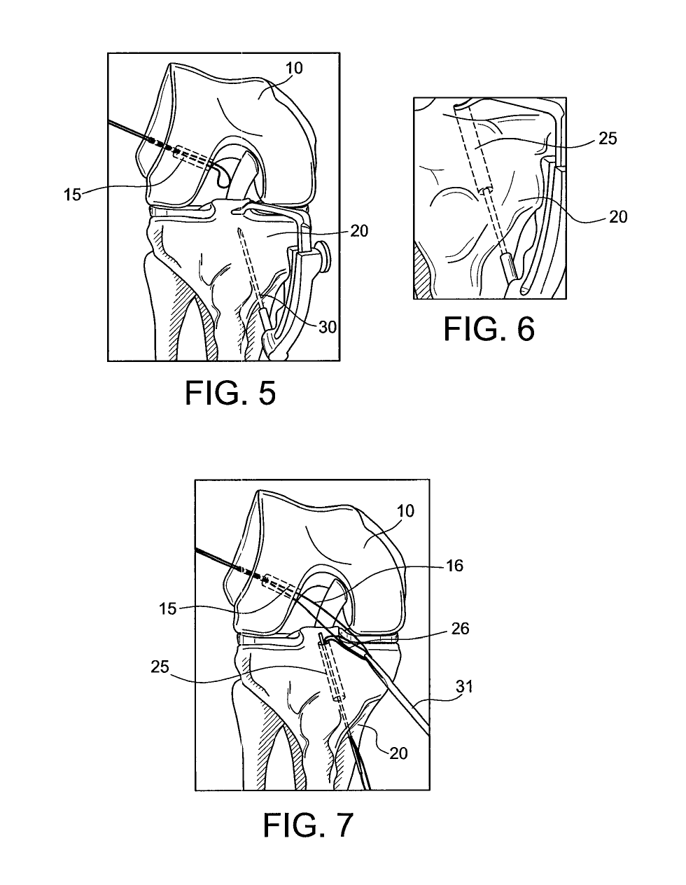

FIGS. 5-7 illustrate tibial socket preparation. Drill the FlippCutter® 30 into the joint and remove the marking hook. Flip the blade and lock into cutting position. Drill on forward, with distal traction, to cut the socket 25. Use the rubber ring and 5 mm markings on the FlipCutter® 30 to measure the socket depth.

Straighten the FlipCutter® blade and remove from the joint. Pass a TigerStick® suture 26 into the joint and retrieve both the tibial TigerStick® 26 and the femoral FiberStick™ 16 out the medial portal together with an open Suture Retriever 31. Retrieving both sutures at the same time will help avoid tissue interposition that can complicate graft passing. A PassPort Button Cannula™ may also be used in the medial portal to prevent tangling. Graft passing sutures from femur and tibia will be docked out of the lateral arthroscopy portal in this step to get them out of the way for the unicondylar knee medial incision.

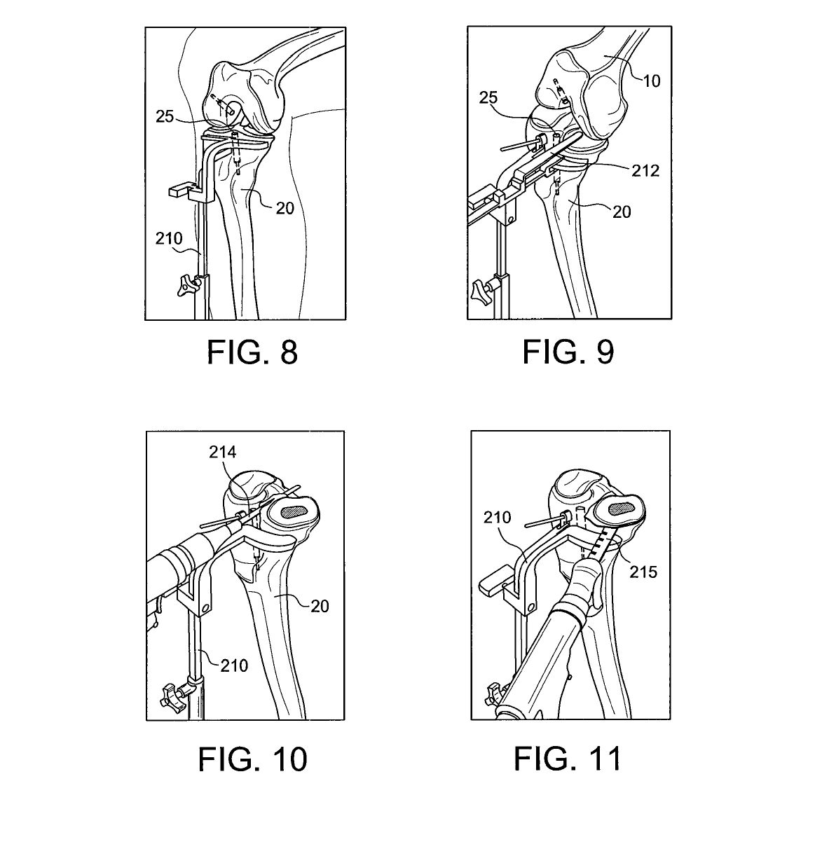

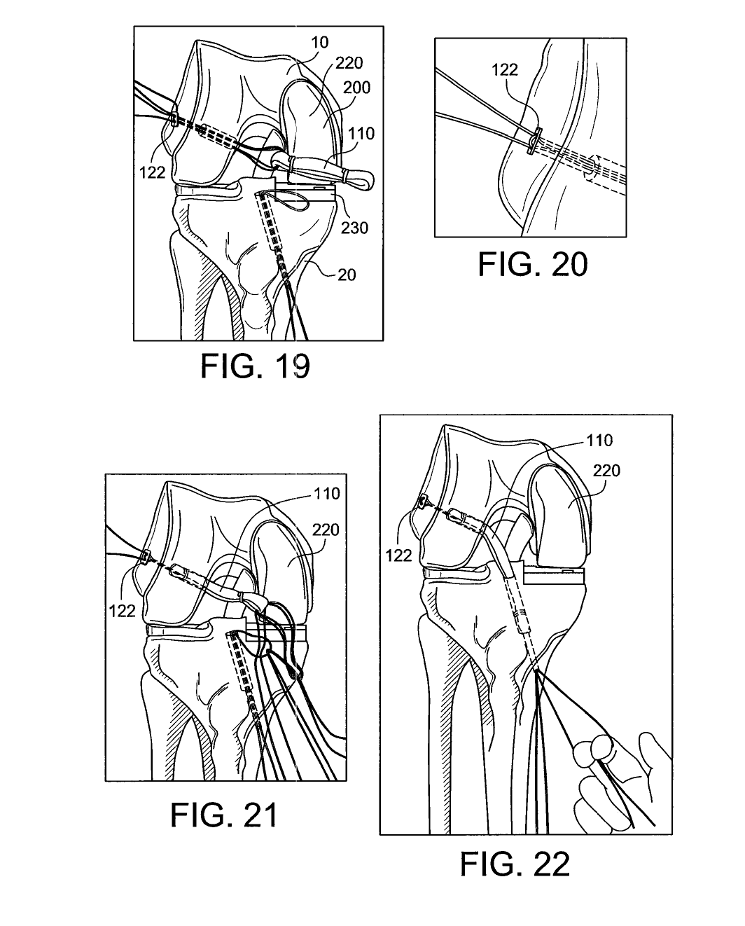

FIGS. 8-18 illustrate the tibial preparation for UKA implant 200 (FIG. 19).

FIG. 8: Assemble the tibial alignment guide 210 and place it on the operative limb. Adjust the length of the tibial alignment guide 210 to allow the proximal portion of the guide to reach the area between the tibial tubercle and the tibial plateau of tibia 20 and pin the guide with a single pin. Varus/valgus alignment is accomplished by aligning the long axis of the tibial guide between the second and third metatarsals and parallel to the long axis of the tibia.

Attach the appropriate (left-medial or right-medial) tibial resection guide to the tibial alignment guide. The curved edge of the tibial resection guide should contact the anterior part of the tibia. The tibial resection should match the anatomic tibial slope. Loosen the screw that allows anterior/posterior motion of the tibial alignment guide relative to the ankle clamp, and adjust the tibial resection guide so that the proximal surface of the tibial alignment guide is parallel to the anatomic slope of the tibia. Tighten the screw to secure tibial slope alignment.

FIG. 9: Tibial Resection Depth: There are at least two options for tibial stylus 212 available. The “0” stylus places the tip of the stylus at the level the saw will cut through, representing zero resection depth. The “5” stylus allows for a 5 mm resection depth below the tip of the stylus. The stylus that is chosen and the resultant tibial resection should be based on the amount of bone loss that is present in the native tibia. Loosen the tibial resection guide thumbscrew so that the resection guide slides proximal/distal on the tibial alignment guide. Slide the tibial stylus over the tibial resection guide and place the stylus pointer on the tibial plateau at the lowest point of the chondral defect. Once the stylus is in the appropriate position and the depth of the cut is determined, lock the tibial resection guide in place on the tibial alignment guide.

The stylus can then be removed from the cutting block. If additional tibial bone should need to be resected, the tibial alignment guide has markings in 1 mm increments that allow the cutting block to be lowered to accommodate additional bony resection.

FIG. 10: Vertical Tibial Cut: Use a reciprocating saw 214 o make the vertical tibial cut. Make the cut parallel to and located at the edge of the tibial eminence from the plateau to the level of the tibial cutting guide guide taking care not to interrupt the tibial tunnel drilled to accept the ACL reconstruction graft. Typically, this cut is made at the mid-point between the top and bottom of the tibial eminence. Use caution to avoid cutting into the ACL attachment. An electrocautery device is helpful to mark the proper orientation line. The location and orientation of the vertical cut will directly influence the size and position of the tibial component. Proper attention to this detail is important for optimal component positioning.

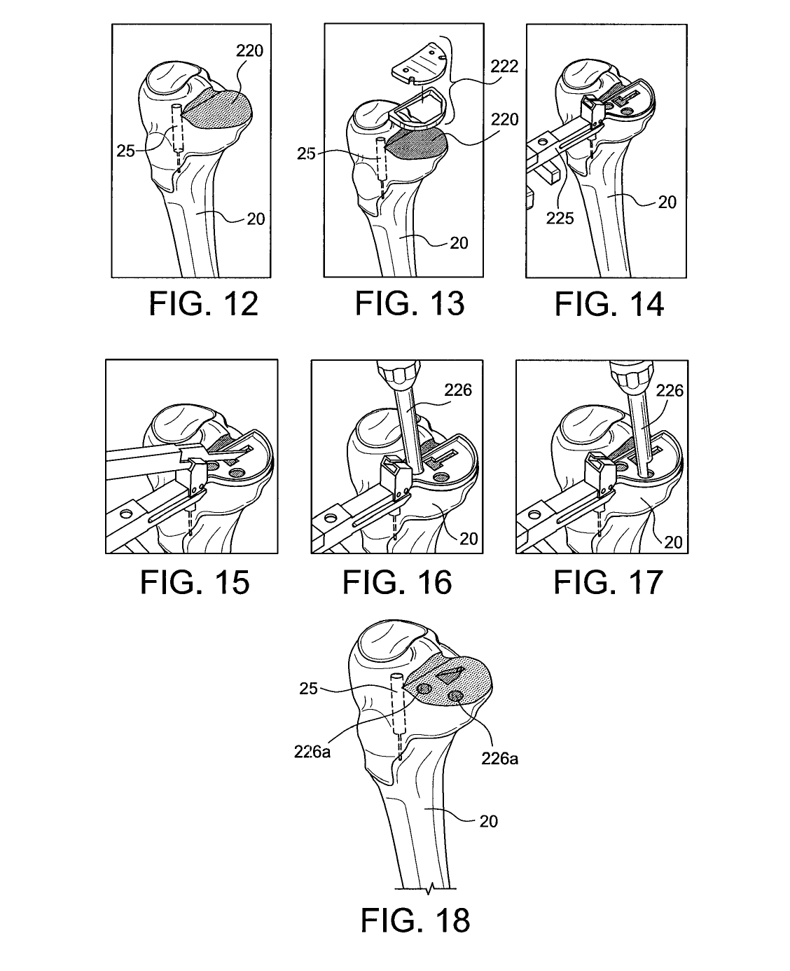

FIGS. 11-12: Horizontal Tibial Cut: Use a saw 215, for example a 1.27 mm×13 mm sagittal saw 215, to make the horizontal cut. Hold the saw blade flat against the surface of the tibial resection guide 210 and take care not to allow the saw to undermine the tibial eminence. Do not flex the blade. Remove the resected tibial plateau with a rongeur or osteotome to obtain cut tibial plateau 220.

Subsequent to the tibial cut, the femoral cut may be conducted and the femur prepared for receiving the femoral component of the UKA implant.

FIG. 13: Trial Reduction: Once the tibial and femoral cuts have been made, use the D-ring tibial trials 222, tibial bearing trials and femoral component trials to assess the fit and position of the implants and the proper tensioning of the compartment. In extension, the joint should be stable but not excessively tight as this can cause the contralateral compartment to be over-stressed. The correct tibial bearing thickness should allow the joint space to open up 1 mm to 2 mm under varus/valgus stress and provide the correction desired but not over-stress the collateral ligaments. If the joint is tight in extension, use a thinner tibial bearing or resect additional distal femoral bone to correct excess tightness.

In flexion, the joint space should also open up 1 mm to 2 mm under stress. Another indicator of excess tightness in flexion is if the tibial bearing trial lifts up anteriorly during flexion. Resect additional posterior femoral bone if the joint is tight in flexion but not extension.

FIGS. 14-18: Tibial Sizing and Finishing: Choose the Tibial Sizing and Finishing Guide 225 that matches the tibial trial used in the previous step. The tibia is sized independent of the prepared femoral size and the exposed tibial bone should be well covered, but there should be no overhang. It is important to remove any peripheral tibial osteophytes to ensure the tibial component is fit to the true cortical rim of the tibia. Insert the guide into the Spurred Handle and lock the guide in the Spurred Handle using the locking screw. Place the guide and Spurred Handle onto the tibia, pushing the spur onto the anterior tibial bone and simultaneously, pushing down onto the tibia to hold the guide in place.

Use the tibial peg step drill 226 to drill the two tibial peg holes 226a. The drill bit can be left in the medial hole to add support during punching the keel. Insert the Keel Punch into the designated slot on the Tibial Guide. Mallet the Keel Punch down into the tibial plateau until it stops. The Keel Punch should be impacted until the tip is flush with the guide.

FIGS. 19-26 illustrate the graft passing and final UKA implant 200 formed of femoral component 220 and tibial component 230. Prior to implanting the graft 100 (GraftLink® construct 100), install the tibial trial bearing into the tibial tray, shuttle the GraftLink® construct in place, wait until the UKA cement dries and then replace the trial bearing with the final poly bearing. Medial UKA femoral implant 220, tibial tray and trial bearing should be in place already before the step of pulling the graft 100 (GraftLink® 100) within the femur and tibia and securing the GraftLink® within the bones, as detailed below with reference to FIGS. 19-26.

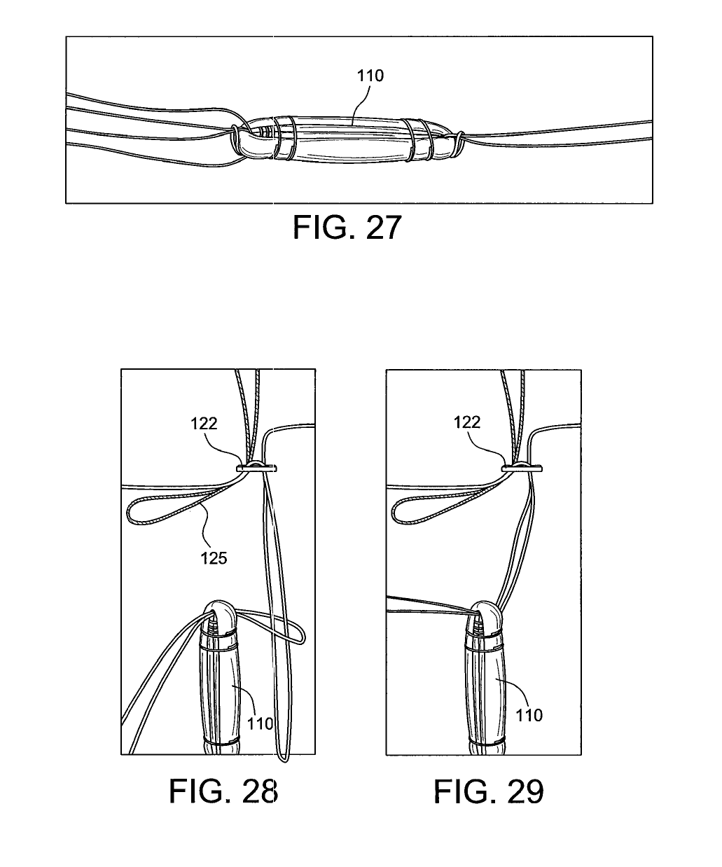

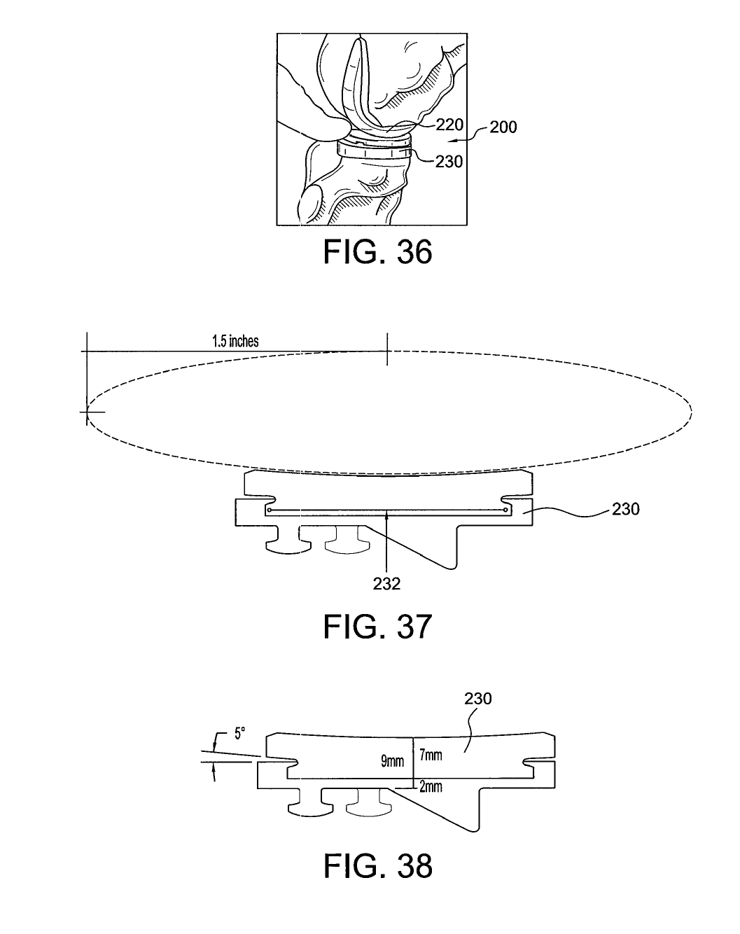

An exemplary graft 100 is shown in FIG. 35 (i.e., an exemplary allograft GraftLink® which is a preconstructed allograft tendon designed to be used with the GraftLink All-inside® ACL technique and TightRope® implants). The presutured construct is designed to be used in the GraftLink® technique to allow for an anatomic, minimally invasive, and reproducible ACL reconstruction. Steps for the formation of exemplary allograft GraftLink® 100 are detailed below with reference to FIGS. 27-35. Details of femoral component 220 and tibial component 230 of UKA implant 200 are shown in FIGS. 36-38.

FIGS. 19-20: Pass the blue button suture and the white shortening strands through the femur. Remove slack from sutures and ensure equal tension. Clamp or hold both blue and white sutures together and pull them together to advance the button out of the femur. Use markings on the loop and arthroscopic visualization of the button to confirm exit from the femoral cortex. Pull back on the graft to confirm the button is seated. While holding slight tension on the graft, pull the shortening strands proximally, one at a time to advance the graft. Pull on each strand in 2 cm increments. The graft 110 can be fully seated into the femur or left partially inserted until tibial passing is complete. The latter option allows fine tuning of graft depth in each socket.

FIG. 21: Cinch a suture around the end of the TightRope® ABS loop to use for passing. Load the cinch suture and the whipstitch tails from the graft into the tibial passing suture. Pull distally on the tibial passing suture to deliver both the TightRope® ABS loop and the whipstitch sutures out of the tibia distally.

FIGS. 22-24: Advance the graft 110 into the tibia 20 by pulling on the inside of the ABS loop and whipstitch sutures. Load the TightRope® ABS button 133 onto the loop. Pull on the white shortening strands to advance the button to bone and tension graft. Ensure the button 133 has a clear path to bone, as to not entrap soft tissue under the button.

FIG. 25: Load the whipstitch sutures into the button 133 and tie a knot 135 for backup fixation.

FIG. 26 illustrates final ACL/UKA repair 500.

FIGS. 27-35 show the formation of an exemplary tissue construct 100 in the form of Allograft GraftLink® 100. The Allograft GraftLink® 100 is a preconstructed allograft tendon designed to be used with the GraftLink® All-inside® ACL technique and TightRope® implants. The graft is typically preloaded with passing sutures to facilitate loading with ACL TightRope® implants. In the example below, a TightRope® is used for femoral fixation and a TightRope® ABS is used for tibial fixation. Alternatively, a TightRope® RT may be used for tibial fixation. Graft 110 may be provided as a presutured tissue graft, for example, presutured allograft, and formed by loading the graft through the implants by folding it symmetrically over the loops and then stitching both graft ends together with a single #2 FiberLoop® after passing the graft through ACL TightRopes. Alternatively, stitching may be conducted on approximately 2 cm of each graft end with one #2 FiberLoop® and one #2 TigerLoop®.

FIGS. 27-32 illustrate loading of the femoral graft end of graft 110 with BTB TightRope® 125. The BTB TightRope® 125 is an open loop construct used on the femoral side of the graft.

FIGS. 28-29: Unfold the blue passing suture of the femoral end of the GraftLink construct, exposing a loop and two tails. Drop the loop of the BTB TightRope® 125 into the blue loop of the passing suture (FIG. 28). Pull the tails of the passing suture to pass the TightRope® loop through the graft 110 (FIG. 29).

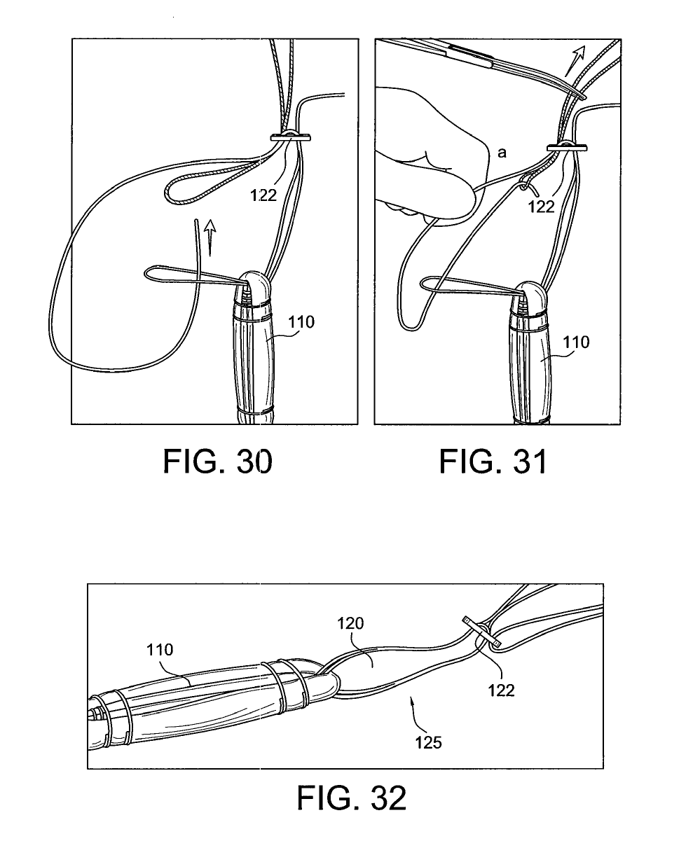

FIGS. 30-32: Pass the free end of the TightRope® implant through the TightRope® loop (FIG. 30). Pass about 2 cm of the free end of the implant through the blue passing suture. While holding the white suture in place, pull proximally on the tails of the blue passing loop until the free end is pinched against the splice of the implant (this will prevent disassembly during passing). Grip the fixed end of the suture (a) with the left hand (FIG. 31). Using a clamp, pull proximally on the blue tails to pass the free end of the implant through the splice and through the TightRope® button 122. Once passed, adjust the loop lengths so that they are equal with the loop connection near the apex of the graft (FIG. 32).

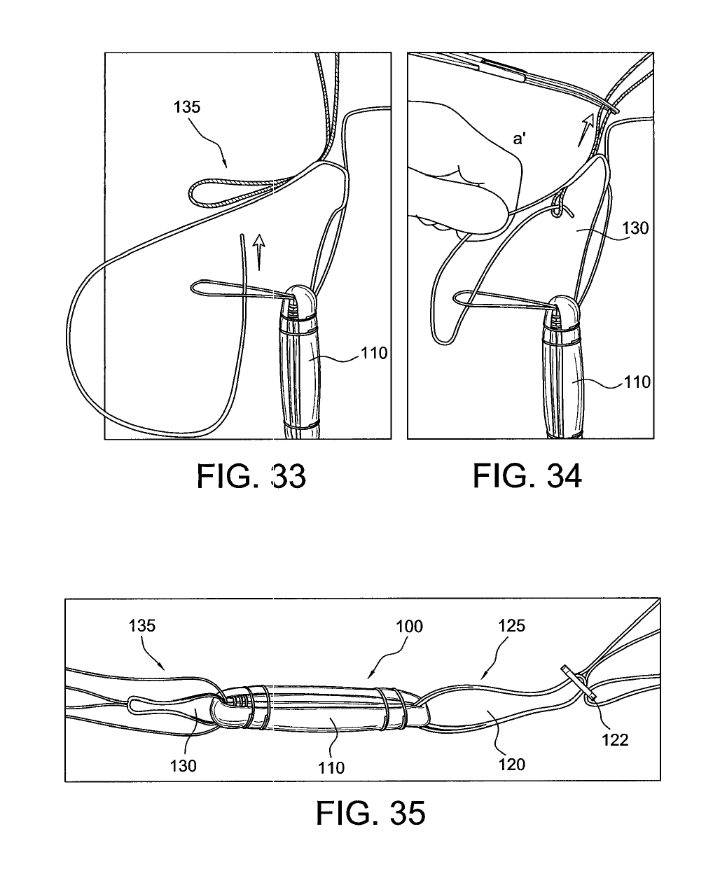

FIGS. 33-35 show loading of the tibial graft end 110 with Open TightRope® ABS 135. An Open TightRope® ABS (Attachable Button System) 135 is used for tibial fixation and loaded onto the graft 110 in similar fashion as the BTB TightRope® 125.

Unfold the blue passing suture of the tibial end of the GraftLink® construct, exposing a loop and two tails. Drop the loop of the TightRope® into the blue loop of the passing suture (FIG. 33). Pull the tails of the passing suture to pass the TightRope® loop through the graft.

Pass the free end of the TightRope® implant through the TightRope® loop (FIG. 34). Pass about 2 cm of the free end of the implant through the blue passing suture. While holding the white suture in place, pull proximally on the tails of the blue passing loop until the free end is pinched against the splice of the implant (this will prevent disassembly during passing). Grip the fixed end of the suture (a′) with the left hand. Using a clamp, pull proximally on the blue tails to pass the free end of the implant through the splice.

FIGS. 36-38 show details of femoral component 220 and tibial component 230 of UKA implant 200. In an exemplary embodiment, at least one of components 220, 230 is formed of polyethylene with a minimum thickness of 6 mm (on the 8 mm poly). The floor of the tibial base plate implant has a thickness of 2 mm. The called out thickness of the polyethylene implants is a combination of the floor thickness of the tibial implant plus the poly thickness (i.e., 8 mm polyethylene implant=2 mm tibial floor+6 mm poly).

The polyethylene implant 230 has a circumferential dovetail 232 that locks into the tibial component. The polyethylene components have a 1.5 inch elliptical radius in the sagittal plane creating an open articulation relationship with the femoral components. The anterior and posterior lip of the polyethylene implant 230 has 5° of clearance built in to allow for ease of insertion.

The methods of the present invention provide surgeons with the instruments and implants to concomitantly perform unicompartmental knee resurfacing and cruciate ligament reconstruction procedures without compromising either implant construct as well as reducing overall morbidities associated with multiple staged surgical procedures

Retrograde drilling, which starts at the level of the resurfacing implants and travels away from the joint line and resurfacing implants allows for more precise placement of the drill holes and also the drill trajectory to avoid undermining the resurfacing implants. Retrograde drills also allow the surgeon to see the drill hole footprint at the joint line and resurfacing implant level more accurately so the surgeon can place the starting point of the drill hole away from the resurfacing implants.

Retrograde cutting and drilling instruments and methods are disclosed, for example, in U.S. Pat. No. 8,652,139 issued Feb. 18, 2014 (disclosing a flip retrograde cutting instrument) or U.S. Pat. No. 8,591,514 issued Nov. 26, 2013 (retrograde cutter with rotating blade) or U.S. Pat. No. 8,038,678 issued Oct. 18, 2011 (dual-sided cutter for forming the femoral trough and tibial socket by retrograde drilling), the disclosure of all of which are incorporated by reference in their entireties herewith.

The rotary drill cutter described in U.S. Pat. No. 8,038,678 is a dual-sided rotary drill cutter that comprises two opposed sides and is provided with cutting surfaces on both sides, such that the rotary drill cutter is configured for cutting in two directions, a cannulation that is threaded such that forward drilling engages the rotary drill cutter to a drill pin, while simultaneously disengaging the cutter from an insertion rod.

The flip cutter instrument detailed in U.S. Pat. No. 8,652,139 is a flip retrograde with a cannulated elongated body having a distal end, a proximal end and a longitudinal axis, the body further comprising a shaft having a blade disposed at its distal end, the blade being securely engaged to the shaft and capable of movement from the straight position to a flip position and vice versa, the blade having a cutting diameter of about 6 mm to about 13 mm, and a locking tube housing the shaft. When the blade is in the straight position, retracting the locking tube allows the blade to articulate and to flip, within the joint space, from the straight position wherein the blade is aligned with the longitudinal axis of the shaft to the flip position which is not aligned with the longitudinal axis of the shaft and with the blade facing the proximal end of the body for retrograde drilling of a bone tunnel or socket. Locking the blade in the flip position by tightening the locking tube allows pulling the retrograde cutter proximally so that the blade in the flip position cuts in a retrograde manner in the bone, from the articular joint space towards an outer surface of the bone, and drills the bone tunnel or socket using the flip retrograde cutter with the blade in the flip position.

The UKA system detailed above is a complete, minimally invasive, instrument and implant platform for the treatment of localized unicondylar cartilage degeneration as a result of osteoarthritis or post-traumatic arthrosis in the medial or lateral compartment of the knee. The UKA system includes highly anatomic femoral and tibial resurfacing implants and a novel and innovative instrument platform that facilitates a highly accurate, efficient and reproducible surgical technique.

The GraftLink® Minimally Invasive ACL Reconstruction technique provides the ultimate in anatomic, minimally invasive and reproducible ACL reconstruction. Its independent tibial and femoral socket preparation with cutters such as the FlipCutter® limits soft tissue dissection, preserves bone/periosteum and facilitates unconstrained placement of the ACL graft in relation to the UKA resurfacing implants without compromising the bone underlying the UKA implants.

As detailed above, the presutured Allograft GraftLink® is a preassembled, sterile allograft tendon that was designed for use with an all-inside ACL technique such as the GraftLink® All-inside® ACL technique. The availability of this presutured allograft provides surgeons with a high quality, consistent, sterile and strong allograft tendon for use in primary or revision ACL procedures and eliminates the time needed to collect and prepare autograft tendon, speeding up the workflow. The tapered graft and adjustable femoral and tibial ACL TightRope® buttons facilitate graft passing, fine tuning of graft depth and graft tensioning from the femoral and tibial sides.

While the present invention is described herein with reference to illustrative embodiments for particular applications, it should be understood that the invention is not limited thereto. Those having ordinary skill in the art and access to the teachings provided herein will recognize additional modifications, applications, embodiments and substitution of equivalents all fall within the scope of the invention.

What is claimed is:

1. A method of knee reconstruction comprising the steps of:
conducting knee ligament reconstruction; and
concomitantly, conducting unicompartment knee arthoplasty.

2. The method of claim 1, wherein the step of conducting knee ligament reconstruction further comprises forming at least a tibial socket in a retrograde manner by drilling from an articular joint surface of tibia and into the tibia.

3. The method of claim 1, further comprising the steps of:
preparing a lateral femoral condyle for securing a femoral implant;
subsequently, forming a femoral socket or tunnel;
subsequently, forming a tibial socket in a retrograde manner using a retrograde drill;
installing a tibial trial bearing into a tibial tray of a tibial implant;
inserting a tissue construct within an articular joint of a knee and securing the tissue construct within the tibial socket and the femoral socket or tunnel;
waiting for cement from the femoral implant to dry; and
replacing the tibial trial bearing with the tibial implant.

4. The method of claim 3, wherein the tissue construct is an ACL construct.

5. The method of claim 3, wherein the tissue construct comprises a ligament and at least one adjustable suture-button for connection to a flexible, adjustable length, continuous loop with two adjustable eyesplices that are interconnected for knotless fixation.

6. The method of claim 5, wherein the adjustable suture-button comprises a first fixation device with slotted ends for connection to the flexible, adjustable length, continuous loop, and a second fixation device in the form of a button.

7. The method of claim 5, wherein the tibial implant is spaced apart from an articular opening of the tibial socket.

8. The method of claim 3, wherein the retrograde drill is a dual-sided rotary drill cutter that comprises two opposed sides and is provided with cutting surfaces on both sides, such that the rotary drill cutter is configured for cutting in two opposite directions.

9. The method of claim 3, wherein the retrograde drill is a flip retrograde cutter comprising a cannulated elongated body having a distal end, a proximal end and a longitudinal axis, the body further comprising a shaft having a blade disposed at its distal end, the blade being securely engaged to the shaft and capable of movement from a straight position to a flip position and vice versa, the body further comprising a locking tube housing the shaft, wherein retracting the locking tube allows the blade to articulate and to flip, within the joint space, from the straight position wherein the blade is aligned with the longitudinal axis of the shaft to the flip position which is not aligned with the longitudinal axis of the shaft.

10. The method of claim 3, wherein the step of forming the tibial socket further comprises:
inserting a flip retrograde cutter through a pre-formed tunnel in tibia and into an articular joint space of the articular knee joint in a straight position, the flip retrograde cutter comprising a cannulated elongated body having a distal end, a proximal end and a longitudinal axis, the body further comprising a shaft having a blade disposed at its distal end, the blade being securely engaged to the shaft and capable of movement from a straight position to a flip position and vice versa, the body further comprising a locking tube housing the shaft;
subsequently, while the blade is in the straight position, retracting the locking tube to allow the blade to articulate and to flip, within the joint space, from the straight position wherein the blade is aligned with the longitudinal axis of the shaft to the flip position which is not aligned with the longitudinal axis of the shaft and wherein the blade faces the proximal end of the body for retrograde drilling of the tibial socket;
locking the blade in the flip position by tightening the locking tube; and
pulling the retrograde cutter proximally so that the blade in the flip position cuts in a retrograde manner in the tibia, from the articular joint space towards an outer surface of the tibia, and drills the tibial socket using the flip retrograde cutter with the blade in the flip position.

11. A method of ligament repair and concomitant unicompartmental knee arthoplasty, comprising the steps of:
drilling a tibial socket or tunnel in a retrograde manner using a flip cutter instrument comprising a shaft and a blade secured to a distal end of the shaft, the blade being configured to flip from a straight position to a flip position and vice versa, the blade advancing, in the flip position, from a joint articular surface of tibia, into the tibia and towards an outer surface of the tibia, by pulling the flip cutter instrument proximally from the articular space and towards the outer surface of the tibia, so that the blade in the flip position cuts in a retrograde manner in the tibia;
installing a tibial trial bearing into a tibial tray secured on the tibia, the tibial trial bearing being spaced from an articular opening of the tibial socket or tunnel;
securing a replacement graft within the tibial socket or tunnel; and
subsequently, replacing the tibial trial bearing with a final tibial implant.

12. The method of claim 11, further comprising the steps of forming a femoral tunnel or socket; preparing a lateral femoral condyle for insertion of a femoral component of a UKA implant; securing the replacement graft within the femoral tunnel or socket; and waiting for cement from the femoral component to dry before the step of replacing the tibial trial bearing with the final tibial implant.

13. The method of claim 12, wherein the step of securing the replacement graft further comprises introducing the replacement graft intraarticularly within a space joint of the knee and then securing the replacement graft within the tibial socket and the femoral tunnel or socket in an all-inside manner.

14. The method of claim 12, wherein the replacement graft is formed of tissue and two fixation systems attached to the tissue, wherein each of the fixation systems comprises an adjustable suture-button for connection to a flexible, continuous loop with two adjustable eyesplices that are interconnected for knotless fixation, the continuous loop having an adjustable length and adjustable perimeter.

15. A system for simultaneous ligament replacement and unicompartmental knee arthoplasty, comprising:
unicompartmental knee arthoplasty tibial and femoral implants;
a retrograde cutter; and
a ligament fixation system comprising a ligament for fixation within a femoral socket and a tibial socket; first and second buttons having different configurations; and two continuous, flexible, adjustable length, loops of flexible material, each of the loops having an adjustable length to allow positioning of the ligament within the tibia and femoral tunnels, and wherein each loop is provided with two discrete splices that allow adjustment in one direction while locking the construct in the opposite direction; and wherein the ligament securely attached to a respective loop for positioning.

16. The system of claim 15, wherein the first button is an oblong button with end slots for connection to the continuous loop, the end slots being laterally arranged to allow attachment to and detachment from the flexible, continuous loop, and wherein the second button is a round button.

17. The system of claim 15, wherein the adjustable loop is formed of a high strength suture.

18. A ligament fixation and UKA implant kit comprising:
first and second buttons, wherein the first button has a plurality of apertures, and the second button has a body with at least one attachment feature disposed on a perimeter of the body, to allow assembly of the second button to a flexible strand;
a flexible coupling mounted, in use, between the first and second buttons, wherein the flexible coupling includes an adjustable, closed, flexible, knotless loop having an adjustable length and two adjustable eyesplices that are interconnected;
a ligament graft attached to the flexible coupling;
a retrograde cutter for removing bone in a retrograde manner; and
femoral and tibial components of a UKA partial knee implant.
